(19) United States
(12) Patent Application Publication
(10) Pub. No.: US 2024/0399032 A1
(43) Pub. Date: Dec. 5, 2024

(54) HYDROGEL FOR PROMOTING GROWTH OF MESENCHYMAL STEM CELLS, AND PREPARATION METHOD AND APPLICATION METHOD THEREOF

(71) Applicants: The Fifth People's Hospital of Hainan Province, Haikou (CN); The Second Xiangya Hospital of Central South University, Changsha (CN)

(72) Inventors: Yan Ding, Haikou (CN); Xiaojie He, Changsha (CN); Tengjiao Xu, Haikou (CN)

(21) Appl. No.: 18/664,310

(22) Filed: May 15, 2024

(30) Foreign Application Priority Data
May 30, 2023 (CN) 2023106281559

(51) Int. Cl. A61L 31/14; A61L 31/16

(52) U.S. Cl. CPC A61L 31/145; A61L 31/16

(57) ABSTRACT

A preparation method of a hydrogel for promoting growth of mesenchymal stem cells (MSCs), and a preparation method and an application method are provided. The hydrogel is rich in MSC-derived exosomes, which can effectively promote wound healing both for diabetic foot ulcer (DFU) caused by bacterial infection and for DFU caused by skin scratch. Moreover, healing speed for the DFU is fast and healing area for the DFU is wide.

### CROSS-REFERENCE TO RELATED APPLICATION

This application claims priority to a Chinese patent application No.2023106281559, filed to China National Intellectual Property Administration (CNIPA) on May 30, 2023, which is herein incorporated by reference in its entirety.

### TECHNICAL FIELD

The disclosure relates to the technical field of medicine, particularly to a hydrogel for promoting growth of mesenchymal stem cells, and a preparation method and an application method thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 24009THXT-USP1-SL.xml. The XML file is 8,192 bytes; is created on May 9, 2024; and is being submitted electronically via patent center.

### BACKGROUND

Diabetic foot ulcers (DFUs), a serious complication of diabetes, are caused by neuropathy (i.e., deficiencies in sensory nerves, motor nerves, and autonomic nerves), ischemia, or an integrated pathogenicity of neuropathy and ischemia. It is reported that prevalence of DFU in diabetics is about 2-10%, but lifetime prevalence of DFU can reach to 25%. According to the prevalence of DFU reported by International Diabetes Federation (IDF) in 2015, 9.1 million to 26.1 million of the diabetics worldwide have foot ulcers every year. Limitation of movement, pain, and discomfort are main common clinical symptoms of DFU, which can lead to amputation in severe cases. In addition, cost of DFU treatment is high. Results of a Chinese study involving 3,654 DFU patients show that the total cost per patient increases from CNY 15535.58 in 2014 to CNY 42040.60 in 2020, with an average of CNY 21826.91. It can be seen that DFU not only affects quality of life of the diabetics, increases their cost of living, but also reduces their life expectancy. The diabetics affected with DFUs have a lower quality of life and poorer psychological and social adaptability, which brings a huge economic burden to their families, society, and healthcare industry.

Studies have found that a mortality rate associated with DFU is approximately 5% in the first 12 months, and a 5-year mortality rate is approximately 42%. At present, conventional clinical treatments for DFUs include control of blood glucose and infection, surgical debridement, application of wound dressings to moisten around the wound and control exudates, local wound decompression, management of peripheral arterial disease, negative pressure therapy, and oxygen therapy, etc., the above of which belong to adjuvant therapies for treating DFU, are beneficial to improve wound healing rate to a certain extent, but are poor in cure rate. Moreover, most of reported data are obtained from randomized controlled trials in a small scale, which has a high risk of bias. Due to complexity and individualized differences of the diabetics affected with DFUs, multidisciplinary collaboration has increasingly become a main treatment for DFUs. However, despite that the diabetics affected with DFUs are treated with the above treatments, the cure rate of DFUs is still very low, and treatment effects for DFUs are urgent to be improved.

Therefore, it is of great significance to develop a drug that can quickly and effectively cure DFUs.

### SUMMARY

In view of the above, an objective of the disclosure is to provide a hydrogel for promoting growth of mesenchymal stem cells (MSCs). The hydrogel for promoting the growth of mesenchymal stem cells is rich in MSC-derived exosomes, which can effectively heal skin affected with diabetic foot ulcer (DFU), and improve healing speed and increase healing area.

In order to achieve the above objective, the disclosure adopts the following technical solution.

On the one hand, the disclosure provides a preparation method of a hydrogel for promoting growth of MSCs, including: using natural raw materials to construct the hydrogel for promoting growth of MSCs, mixing the hydrogel with MSC-derived exosomes to obtain a sustained-release preparation, and administrating the sustained-release preparation on a wound affected with DFU.

On the other hand, the disclosure provides a hydrogel for promoting growth of MSCs obtained from the above-mentioned preparation method of the hydrogel for promoting growth of MSCs.

On the still other hand, the disclosure provides an application method of the hydrogel for promoting growth of MSCs, including: preparing a drug for healing skin affected with DFU.

Beneficial effects of the disclosure include that the hydrogel for promoting growth of MSCs provided by the disclosure can effectively promote wound healing, and has fast healing speed and large healing area, whether DFU is caused by bacterial infection or is caused by skin scratch.

### BRIEF DESCRIPTION OF DRAWINGS

FIG. 1 illustrates a wound of a Staphylococcus aureus model rat.

FIG. 2 illustrates a wound of a skin scratch model rat.

FIG. 3 illustrates a wound of the skin scratch model rat combined with glacial acetic acid on the wound.

FIG. 4 illustrates wound healing of a Staphylococcus aureus model rat of H4 group treated with mecobalamin.

FIG. 5 illustrates wound healing of a Staphylococcus aureus model rat of T10 group treated with a hydrogel.

FIG. 6 illustrates wound healing of a Staphylococcus aureus model rat of T11 group treated with the hydrogel.

FIG. 7 illustrates wound healing of a skin scratch model rat of H1 group treated with the mecobalamin.

FIG. 8 illustrates wound healing of a skin scratch model rat of M1 group treated with epalrestat.

FIG. 9 illustrates wound healing of a skin scratch model rat of T1 group treated with the hydrogel.

FIG. 10 illustrates wound healing of a skin scratch model rat of T2 group treated with the hydrogel.

FIG. 11 illustrates wound healing of a skin scratch model rat of T3 group treated with the hydrogel.

FIG. 12 illustrates wound healing of a skin scratch model rat of T4 group treated with the hydrogel.

FIG. 13 illustrates wound healing of a skin scratch model rat combined with the glacial acetic acid of M2 group treated with the epalrestat.

FIG. 14 illustrates wound healing of a skin scratch model rat combined with the glacial acetic acid of T5 group treated with the hydrogel.

FIG. 15 illustrates wound healing of a skin scratch model rat combined with the glacial acetic acid of T6 group treated with the hydrogel.

FIG. 16 illustrates wound healing of a skin scratch model rat combined with the glacial acetic acid of T7 group treated with the hydrogel.

FIG. 17 illustrates wound healing of a skin scratch model rat combined with the glacial acetic acid of T8 group treated with the hydrogel.

FIG. 18 illustrates expression of hypoxia inducible factor 1 subunit alpha (Hif-1α) on the wound of the Staphylococcus aureus model rat up-regulated by the hydrogel.

FIG. 19 illustrates secretion expression of vascular endothelial growth factor (VEGF) of the Staphylococcus aureus model rat up-regulated by the hydrogel.

FIG. 20 illustrates secretion expression of stromal cell-derived factor-1 (SDF-1α) of the Staphylococcus aureus model rat up-regulated by the hydrogel.

FIG. 21 illustrates secretion expression of platelet derived growth factor-beta (PDGF-β) of the Staphylococcus aureus model rat up-regulated by the hydrogel.

### DETAILED DESCRIPTION OF EMBODIMENTS

The illustrated embodiments aim to better explain the disclosure, but the content of the disclosure is not limited to the illustrated embodiments. Therefore, those skilled in the related art are able to make non-essential improvements and adjustments to an implementation scheme based on the above content of the disclosure. However, the improvements and adjustments still fall within the scope of the protection of the disclosure.

Terms used herein are only intended to describe specific embodiments and are not intended to limit the disclosure. Unless the terms have clearly different meanings in the content of the disclosure, singular expressions include plural expressions. As used herein, it should be understood that the terms such as “including”, “having”, “containing” are intended to indicate existence of features, numbers, operations, components, parts, elements, materials, or combinations. The terms of the disclosure are disclosed in the specification and are not intended to exclude a possibility of the existence or addition of one or more other features, numbers, operations, components, parts, elements, materials, or combinations thereof. As used herein, “/” can be interpreted as “and” or “or” depending on actual situation.

An embodiment of the disclosure provides a preparation method of a hydrogel for promoting growth of mesenchymal stem cells (MSCs), including the following steps: using natural raw materials to construct the hydrogel for promoting growth of MSCs, mixing the hydrogel with MSC-derived exosomes to obtain a sustained-release preparation, and administrating the sustained-release preparation on a wound affected with diabetic foot ulcer (DFU). Specifically, the disclosure synthetizes the natural raw materials in vitro, which includes: beta-glycerophosphate (β-GP), chitosan (CS), and methyl cellulose (MC). The β-GP, the CS, and the MC are mixed according to a certain proportion to obtain a thermosensitive injectable three-dimensional (3D) hydrogel. Thereafter, the MSC-derived exosomes (also referred to as MSC-exosomes) secreted by the extracted and purified MSCs can be mixed with the hydrogel (i.e., the thermosensitive injectable three-dimensional (3D) hydrogel) to make the 3D hydrogel carried with the MSC-derived exosomes slow release at the wound affected with DFU.

The CS is a polysaccharide derived from chitin by deacetylation reaction, and the CS has beneficial effects of biocompatibility, biodegradability, non-toxicity, and promoting wound healing. Therefore, the CS has long been used as a basic scaffold for drug delivery and has broad application value in the field of biomedicine. The β-GP is a sodium salt, which can make the CS form the thermosensitive injectable 3D hydrogel. The MC is added into the thermosensitive injectable 3D hydrogel to enhance strength of the hydrogel. The specific preparation method of the hydrogel is as follows: the CS, the β-GP, and the MC powder are sterilized by ultraviolet (UV), and the following three stock solutions are prepared: 35% (referred as to a mass concentration calculated by w/v) CS stock solution, 56% (w/v) β-GP stock solution, and 10% (w/v) MC stock solution. Thereafter, the CS stock solution, the β-GP stock solution, and the MC stock solution are mixed evenly according to a proportion of 2:1:0.1 to obtain mixed solution, and then the extracted solution of MSC-derived exosomes or the growth factor solution synthesized in vitro is added into the above-mentioned mixed solution according to a proportion of 10% of a total volume of the above-mentioned mixed solution, thereafter mixing evenly to obtain prepared solution. Finally, the prepared solution is placed still in water bath at 37 degrees Celsius (C) for 3 minutes to form the hydrogel containing the MSC-derived exosomes.

It should be noted that the hydrogel is a stable hydrophilic network cross-linked structure through physical, chemical, and biological enzymatic cross-linking, with good water retention, biocompatibility, responsiveness, and degradability. The hydrogel has shown great application potential in clinical medicine, and can be used as a drug-controlled release carrier, a tissue filling material, an artificial vitreous body (AVB), an artificial cartilage, a medical excipient, a drug disintegrant, a corneal contact lens material, a medical cosmetic material, and analysis and medical diagnosis. When the hydrogel is applied to the treatment of skin wound, it has many advantages such as preventing formation of scab; creating a low oxygen environment to promote capillary formation, and to release and activate various growth factors; facilitating dissolution of fibrin and necrotic tissue; being replaced without discomfort because of no adhesion on newly generated granulation tissue; reducing the number of dressing changes, alleviating wound pain, and reducing the scar formation.

The MSCs are multipotent stem cells and belong to an important member of stem cell family, as well as are regarded as adult stem cells (ASC). The MSCs have advantages of easy acquisition, multiple differentiation potentials, good proliferation rate, and clinical safety, so that they are widely used in treatments of various diseases. The MSC-derived exosomes are bioactive substances secreted by the MSCs cultured in a serum-free medium (SFM) without animal-derived MSCs, containing a wide variety of lipids, deoxyribonucleic acid (DNA), messenger ribonucleic acid (mRNA), proteins, and carbohydrates. The MSC-derived exosomes have many advantages, such as low immunogenicity, stable content and easy preservation, and cellular immunity improvement by regulating extracellular environment and drug delivery. Moreover, the extraction and purification methods of the MSC-derived exosomes are relatively mature, and it is easy to achieve mass production of the MSC-derived exosomes. Therefore, the MSC-derived exosomes are chosen as the drug delivered by the hydrogel in the disclosure to realize the treatment of the wound for the diabetic.

When the MSC-derived exosomes are smeared on surface of the skin, the exosomes are rapidly cleared because of bodily fluids or friction, so that the repair effect of the MSC-derived exosomes on skin damage is limited. In recent years, research has found that the combined application of hydrogel and stem cell-derived exosomes to skin wound repair can play the functions of both simultaneously and synergically promote skin wound healing. Vajihe Taghdiri Nooshabadi et al. (Vajihe Taghdiri Nooshabadi et al., “Impact of exosome-loaded chitosan hydrogel in wound repair and layered dermal reconstitution in mice animal model”, Journal of Biomedical Materials Research, 2020 Nov. 1, p. 2138-2149, Vol. 108, No. 11.) found that a chitosan hydrogel rich in the MSC-derived exosomes can not only promote in vitro migration and proliferation of fibroblasts, but also promote wound closure and re-epithelialization. In recent years, many studies have shown that hydrogels rich in the MSC-derived exosomes can promote the healing effects of skin wounds for the diabetic and the skin affected with DFU. Jiayi Yang et al. (Jiayi Yang et al., “Umbilical Cord-Derived Mesenchymal Stem Cell-Derived Exosomes Combined Pluronic F127 Hydrogel Promote Chronic Diabetic Wound Healing and Complete Skin Regeneration”, International Journal of Nanomedicine, 2020 Aug. 11, p. 5911-5926, Vol. 15) found that umbilical cord MSC-derived exosomes combined with Pluronic F127 hydrogel promote wound healing and complete skin regeneration in chronic diabetes. Sojin Shin et al. (Sojin Shin et al., “MicroRNA-513a-5p mediates TNF-α and LPS induced apoptosis via downregulation of X-linked inhibitor of apoptotic protein in endothelial cells”, Biochimie, 2012 June, p. 1431-6, Vol. 94, No. 6) found that the hydrogel rich in the MSC-derived exosomes can significantly promote in vitro effects on proliferation, migration and tube formation of human umbilical vein endothelial cells (HUVECs), and significantly improve healing efficiency of full-thickness skin (also referred to full skin) wound of the diabetic in vivo, which is characterized by improved wound closure rate, rapid angiogenesis, re-epithelialization and collagen deposition on the wound, and has a significantly better healing result than smearing the MSC-derived exosomes or the hydrogel alone on the wound.

Based on the above, the disclosure develops a new hydrogel for promoting growth of MSCs that can promote healing of the wound affected with DFU.

In addition, the MSCs are multifunctional, non-hematopoietic adult stem cells that express surface markers with CD90, CD105, and CD73 (cell surface proteins) but do not express surface markers with CD14, CD34, and CD45. The MSCs can differentiate into a mesenchymal cell line, including osteoblasts, chondrocytes, adipocytes, endothelial cells, and cardiomyocytes, as well as a non-mesenchymal cell line, such as hepatocytes and neuron cells. In addition to the differentiation potentials, the MSCs are also capable of secreting nutritional factors such as growth factors and cytokines, as well as extracellular vesicles. The MSCs have become a promising cell therapy for treating human diseases due to their differentiation, self-renewal, and immune regulatory abilities, and have received widespread attention. A large number of studies have confirmed the potential of MSCs in the treatment of human diseases, such as diabetes, cancer, diseases of liver, bone, cartilage, brain, and cardiovascular disease. However, due to the negative effects of donor age and long-term culture on differentiation and proliferation of the MSCs, as well as tumorigenesis of the MSCs, the clinical application of MSCs has been limited. Therefore, it is necessary to explore a new alternative strategy to develop the therapeutic potential of MSCs while eliminating complications of cell transplantation. Recently, it has been found that the therapeutic effect of MSCs is mainly related to paracrine of certain molecules contained in the extracellular vesicles, such as proteins, lipids, mRNA, and microRNAs. Therefore, the extracellular vesicles can reduce the risk of transplanted MSCs differentiating into erroneous cells in response to the local environment, while preserving the beneficial therapeutic effects of paracrine of the MSCs. Furthermore, the extracellular vesicles can minimize risks of donor stem cell rejection and tumor formation, as well as facilitate storage and transportation.

Exosomes belong to a subset of the extracellular vesicles with a diameter range of 40-200 nanometers abbreviated as nm (average 100 nm) enclosed by lipid bilayer membranes that are secreted by most eukaryotic cells. The exosomes play a major role in intercellular signaling by transferring their contents, including proteins, lipids, and nucleic acids. The MSC-derived exosomes exhibit excellent repair effects in various tissue injuries, such as liver, cardiovascular, and skin injuries, and involve mechanisms of angiogenesis, cell proliferation regulation, and immune regulation. The MSC is substituted by the MSC-derived exosomes, which has become a new strategy for tissue regeneration. Several studies have shown that the MSC-derived exosomes can be used in the treatment of diabetic foot. Moreover, the MSC-derived exosomes promote healing of the wound affected with DFU by regulating the inflammatory microenvironment of wound, promoting angiogenesis, and realizing antioxidant activity and cell apoptosis.

Another embodiment of the disclosure provides a hydrogel for promoting growth of MSCs, which is obtained by the above-mentioned preparation method.

Still another embodiment of the disclosure provides an application method of the hydrogel for promoting growth of MSCs, including: preparing a drug for healing skin affected with DFU.

In some illustrated embodiments, the application method further includes: up-regulating an expression of hypoxia inducible factor 1 subunit alpha (Hif-1α) in the DFU by using the drug prepared by the hydrogel for promoting growth of MSCs.

In some illustrated embodiments, the application method further includes: accelerating secretions of vascular endothelial growth factor (VEGF), stromal cell-derived factor-1 (SDF-1α), and platelet derived growth factor-beta (PDGF-β) by using the drug prepared by the hydrogel for promoting growth of MSCs.

It should be noted that the hydrogel for promoting growth of MSCs according to the disclosure can improve the expression of Hif-1α in the DFU and accelerate the secretions of VEGF and SDF-1α and PDGF-β, thereby changing phenotype and gene expression profile of angiogenesis related cells and immune cells, repairing microvascular damage, and ultimately enhancing healing effects of the skin affected with DFU.

It should also be noted that the DFU is caused by neuropathy, ischemia, or an integrated pathogenicity of neuropathy and ischemia. Specially, peripheral neuropathy and ischemia or neuroischemic lesions are initiating factors of the DFU, while infection is usually secondary. Therefore, the DFU has characteristics such as peripheral neuropathy, vascular damage (arterial circulation), inflammatory cytokine infiltration, and susceptibility to infection. As one of the most serious complications for the diabetics, the main cause of DFU is impairment of angiogenesis, and the impairment of microvascular formation and expansion is the main reason for the difficulty of wound healing in the diabetics.

Wound healing is a complex process that includes hemostasis, inflammation, proliferation, and remodeling. During normal wound healing, angiogenesis relies on a subtle balance between promoting vascular growth and proliferation and promoting vascular maturation and quiescence. Diabetes can significantly disturb the subtle balance and destroy proper wound healing; and the interrupted subtle balance leads to hypoxia. The hypoxia is an important activator of endothelial cells around the wound and adjacent vascular systems. Systemic microangiopathy delays cell infiltration, collagen synthesis, angiogenesis, granulation tissue formation, and re-epithelialization due to insufficient transfers of oxygen and nutrients. The normal capillary network is essential to deliver the oxygen, nutrients, and growth factors needed for the wound healing. Therefore, reconstruction of the capillary network is of great importance for the wound of the diabetics. According to a research, microvascular responses to slight thermal injury in view of foot skins of 23 type I diabetics as well as 21 healthy people as a control group are detected by laser Doppler flowmetry (LDF), and it is found that an average maximum blood flow for the skin of the diabetics is significantly lower than that of the control group, thus indicating that the capillary located at the diabetics' skin cannot respond normally to the wound, which may be an important factor in the formation of DFU after minor wound. Another research also shows that the microvascular response of diabetics with microvascular complications to mechanical wound is impaired, which may lead to infection and poor wound healing.

It should also be noted that in an early stage of the skin affected with DFU, hypoxia is an important stimulus for the wound healing and induces blood vessels to generate related cytokines such as expressions of VEGF, SDF-1α, and PDGF-β. The VEGF and the PDGF-β are crucial for vascular development, which can increase endothelial cell proliferation, survival, and migration, and promote angiogenesis; while the SDF-1α can increase angiogenesis by recruit circulating endothelial progenitor cells (EPCs). Previous studies have confirmed that the VEGF, the PDGF-β, and the SDF-1α are regulated by the Hif-1 that is crucial in regulating cellular oxygen homeostasis and adaptive response to the hypoxia. In an early stage of the normal wound healing, Hif-1α can be highly expressed in the wound, while in view of the diabetics, function of the Hif-1α can be inhibited by p300 (referred as to an antibody) induced by high glucose and modified by reactive oxygen species (ROS), leading to a decrease in the vascular network formation. And over time, the ulcers gradually form, and once the ulcers form, they are difficult to reverse.

In addition, next-generation sequencing (NGS) is rapidly developed, which provides valuable insights into complex biological systems. Based on genomics, transcriptomics, and epigenomics of the NGS, the public is increasingly concerned about the characteristics of individual cells. Single-cell RNA sequencing (scRNA-seq) can reveal complex and rare cell populations, reveal regulatory relationships between genes, and track trajectories of different cell lines during development. In recent years, many researches have studied various cell phenotypes and antigen specificity of diabetes by using the scRNA-seq. Georgios Theocharidis et al. (Georgios Theocharidis et al., “Single cell transcriptomic landscape of diabetic foot ulcers”, Nature Communications, 2022 Jan. 10, page 181, Vol. 13, No. 1) analyze 174,962 single cells obtained from foot, forearm, and peripheral blood monocytes of the diabetics affected with DFU by using the scRNA-seq, founding that in the healed wounds of the diabetics affected with DFU is accompanied with overexpression of the Hif-1α, leading to increased populations of unique fibroblast and increased polarization of M1 macrophages. Furthermore, a research has found that the M1 macrophages are more abundant in the healed diabetics affected with DFU and M2 macrophages are more abundant in non-healed diabetics affected with DFU, thus identifying the cell types crucial for promoting the wound healing of the diabetics affected with DFU and potentially providing new treatment methods for DFU treatment. Georgios Theocharidis et al. (Georgios Theocharidis et al., “Integrated Skin Transcriptomics and Serum Multiplex Assays Reveal Novel Mechanisms of Wound Healing in Diabetic Foot Ulcers”, Diabetes, 2020 October, p. 2157-2169, Vol. 69, No. 10) also finds by using scRNA-seq that, compared with the control group, the back skins of the diabetics (also referred as to patients with diabetes mellitus abbreviated DM) and the diabetics' specimens affected with DFU have multiple fibroblast clusters and increased inflammation; interleukin-13 and interferon-γ in myeloid cells of the patients with DM are inhibited and biological processes are disturbed; and migration characteristics of immune cells are impaired. Moreover, genes of solute carrier organic anion transporter family member 2A1 (SLCO2A1) and cytochrome P450 family 1 subfamily A Member 1 (CYP1A1) are mainly expressed by the endothelial cell clusters in the DFU, which facilitates discovering individual genes and pathways that contribute to promoting DFU healing.

In order to better understand the disclosure, the content of the disclosure will be further elaborated with illustrated embodiments, but the content of the disclosure is not limited to the following embodiments.

In the following embodiments, drugs used in the disclosure are illustrated in the following Table 1.

TABLE 1
Drugs used in the disclosure

| Name | Trademark | Specification | | Item No. |
|---|---|---|---|---|
| Streptozocin (STZ) | Solarbio ® | 1 | gram (g) | S8050 |
| Epalrestat | DYNE PHARMA ® | 50 | milligrams (mg) | H20210893 |
| Mecobalamin | Methycobal | 0.5 | mg | H20143107 |
| Staphylococcus aureus | SHBCC ® | — | | ATCC6538 |
| Glacial acetic acid | Sinopharm ® | 500 | milliliters (mL) | FA3550002 |
| Sodium citrate | Xiya Reagent ® | 500 | g | A10231 |
| Trisodium citrate dihydrate | Xiya Reagent ® | 500 | g | A12150 |

In the following embodiments, instruments and equipment used in the disclosure are illustrated in the following Table 2.

TABLE 2
Information on instruments and equipment used

| Instrument | Company | Item No. |
|---|---|---|
| Refrigerator | Haier | — |
| Water purification system | Bio-Rad | Master Evo- |
| (Type III water used in lab) | | S45UVF |
| Air-conditioner | Yangzi ® | — |
| Shake | Haimen Kylin-Bell Lab | KB-700 |
| | Instruments Co., Ltd | |
| −80° C. refrigerator | Anhui Zhongke Duling | MDF-86V588D |
| | Commercial Electrical | |
| | Appliance Company | |

In the following embodiments, STZ solution used in the disclosure is prepared as illustrated in the following Table 3.

TABLE 3
Preparation of STZ solution

| Name | Solvent | Dissolution method |
|---|---|---|
| A-citric acid | Sterile water | 2.1 g/100 mL |
| B-trisodium citrate | Sterile water | 2.94 g/100 mL |
| dihydrate | | |
| C-citrate buffer | A + B | 1 mL of solution A + 1.32 mL |
| | | of solution B |
| STZ | C | 50 mg/kg of STZ dissolving |
| | | 100 microliters (μL) of C/rat |

### 1. Construction of DM Rat Model

30 specific pathogen free (SPF) Sprague Dawley (SD) male rats aged 6-8 weeks old (weight 190 g or so) are used to construct as the DM rat model. Specifically, the STZ solution is prepared and used, and the prepared STZ solution is used within 10 minutes. The STZ solution is injected into left lower abdomens of the rats with a single dose of 55 milligrams per kilogram (mg/kg). After a week of the intraperitoneal injection of the STZ solution, the rats are in abrosia but can have water for 12 hours (h), thereafter blood glucose of the rats is detected, and the rats with blood glucose value above 12.6 millimoles per milliliter (mmol/mL) are selected as the DM rat model. The 30 SPF SD male rats are all constructed as the DM rat model, 12 rats died before diabetic foot administration, and 2 rats died during the administration but not appearing effectiveness.

2. Construction of DM Rat Model with Diabetic Foot (DF)
(1) Modeling of Staphylococcus aureus Suspension

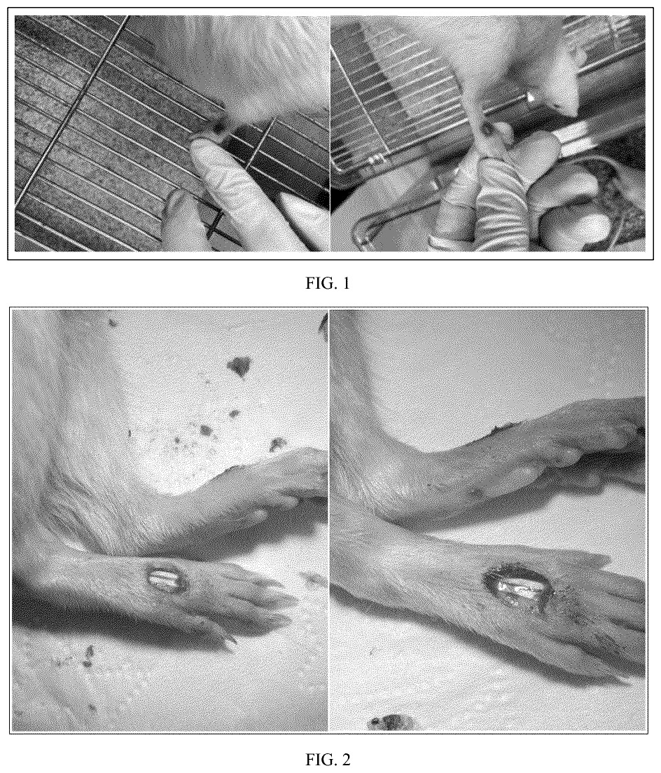

Six DM model rats are selected. At the third week, dorsal sides of hindfoots of the DM model rats are subcutaneously injected 10 μL of suspension containing more than 106 Staphylococcus aureus, thereby obtaining a DM animal model with foot infection (also referred to as extremity infection). Subsequently, the diabetic foots of the DM animal model are observed daily, as shown in FIG. 1. It can be seen from FIG. 1, on the sixth day, spontaneous foot gangrene occurs, and on the seventh day and eighth day, the foot gangrene is gradually obvious.

### (2) Modeling of Skin Scratch

Six DM model rats are selected for normal feeding for four weeks. After anesthesia, round skin wounds with each diameter of 5 mm are created on dorsal sides of hindfoots of the DM model rats to generate foot gangrene, as shown in FIG. 2. And then, the DM model rats continue to be fed regularly and the skin wounds of the DM model rats are observed every day.

(3) Modeling of skin scratch combined with glacial acetic acid

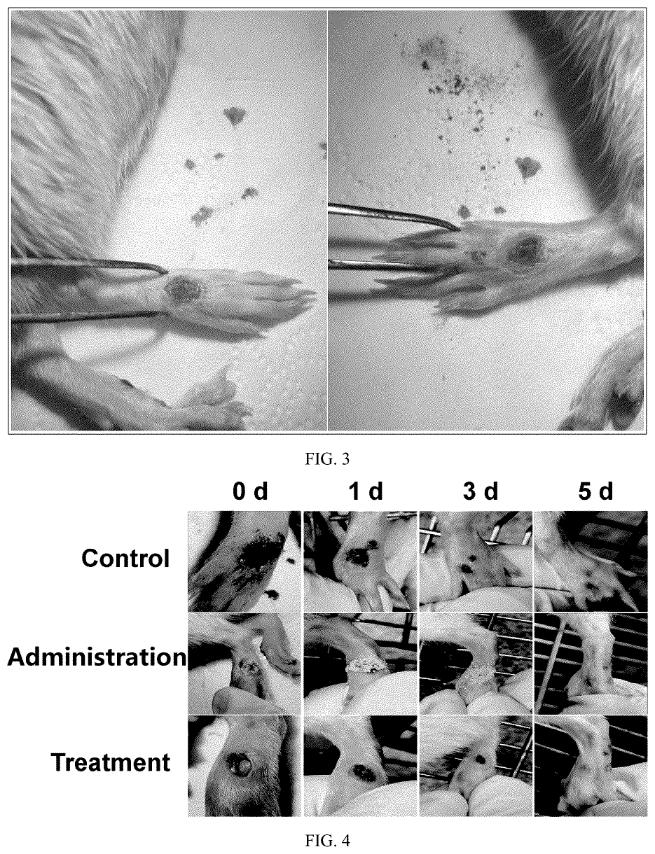

Six DM model rats are selected for normal feeding for four weeks. After anesthesia, round skin wounds with each diameter of 5 mm are created on dorsal sides of hindfoots of the DM model rats. Simultaneously, the round skin wounds are wiped by using the glacial acetic acid with a concentration of 50% once a day for a continuous week. As shown in FIG. 3, the skin wounds are observed daily.

3. Treatment for DM Rat Model with DF
(1) Treatment for Staphylococcus aureus Model Rat

Therapeutic drug of mecobalamin (H4 group) is applied daily on the wound disposed on the left hindfoot of the Staphylococcus aureus model rat, and then the wound of the model rat is observed. The wound is shown in FIG. 4. After 5 consecutive days, the treated wound and the untreated wound both heal, and no significant difference is observed.

Therapeutic drug of epalrestat (M4 group) is applied daily on the wound disposed on the left hindfoot of the Staphylococcus aureus model rat.

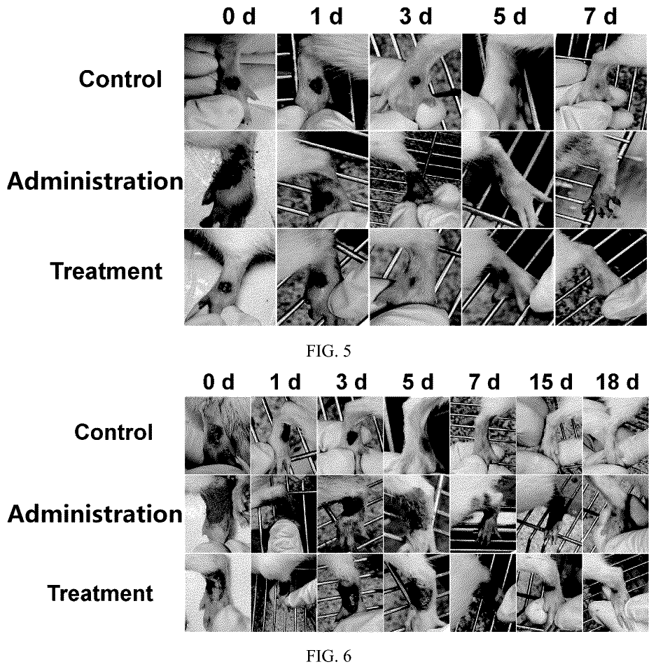

The hydrogels (T10 group and T11 group) are applied daily on the wounds disposed on the left hindfoots of four Staphylococcus aureus model rats, and then the wounds of the 4 model rats are observed. The wound of the T10 group is shown in FIG. 5. The healing speed of the foot gangrene of the model rat treated with the hydrogel is significantly faster than that of the control group under the condition that areas of the foot gangrenes are the same. Moreover, the foot gangrene healed after 5 days of administration, and the control group healed after 7 days. The wound of the T11 group is shown in FIG. 6, which illustrates the foot gangrene of the control group healed after 7 days, and there is slight scabbing on the foot gangrene of the model rat treated with the hydrogel after 18 days. It can be seen that the area of foot gangrene affects the healing speed of the drug administrated on the wound.

(2) Treatment for Skin Scratch Model Rat

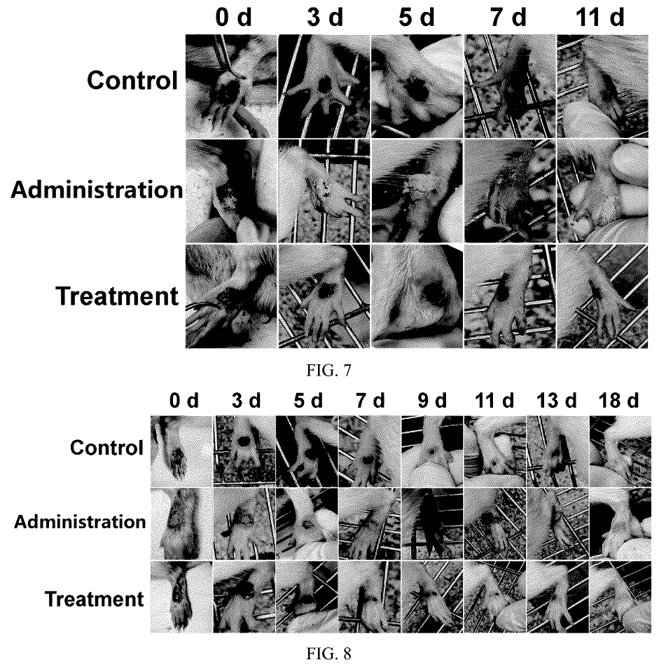

Therapeutic drug of mecobalamin (H1 group) is applied daily on the wound disposed on the left hindfoot of the skin scratch model rat, and then the wound of the model rat is observed. The wound is shown in FIG. 7. The wound healed even without administration, at the same time, the wound of the skin scratch model rat treated with drug also healed after 11 days, indicating that the drug has no significant promoting effect on the wound healing.

Therapeutic drug of epalrestat (M1 group) is applied daily on the wound disposed on the left hindfoot of the skin scratch model rat, and then the wound of the model rat is observed. The wound is shown in FIG. 8. After 13 days of continuous administration, the wound healed, and at the same time, the wound of the skin scratch model rat without administration healed on the eighteenth day. Therefore, the drug has a promoting effect on wound healing.

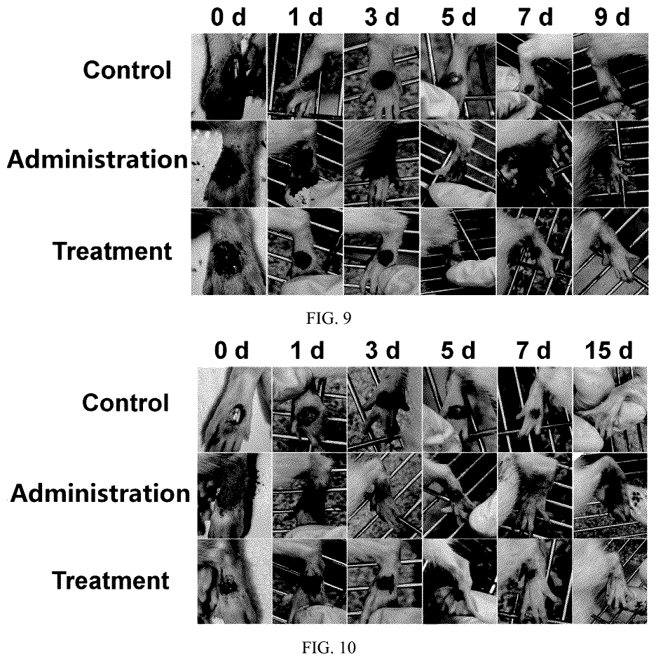

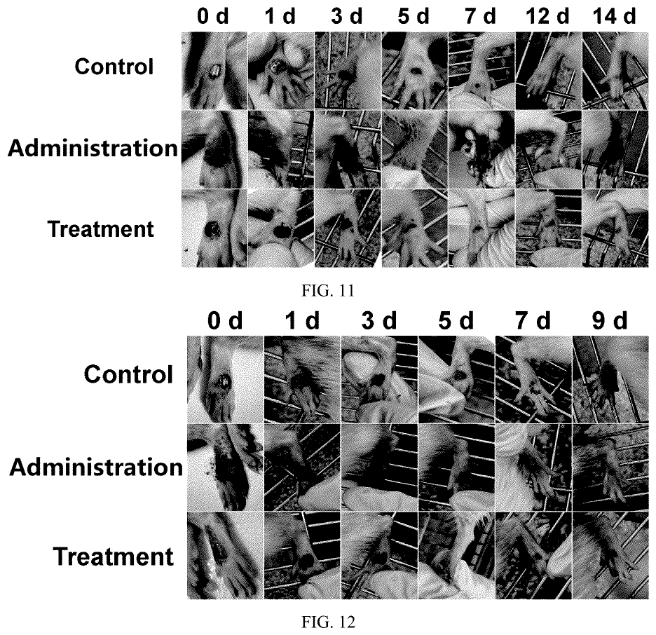

The hydrogels (T1 group, T2 group, T3 group, T4 group) are applied daily on the wounds disposed on the left hindfoots of four skin scratch model rats, and then the wounds of the 4 model rats are observed. The wound of the T1 group is shown in FIG. 9, the wound of the T2 group is shown in FIG. 10, the wound of the T3 group is shown in FIG. 11, and the wound of the T4 group is shown in FIG. 12. The above results show that the healing speed of two foots with the administration is faster than those of the control group, and the healing speed of two foots with the administration is the same as those of the control group, indicating that the hydrogel has the effect of promoting foot gangrene healing.

(3) Treatment for Skin Scratch Model Rat Combined with Glacial Acetic Acid

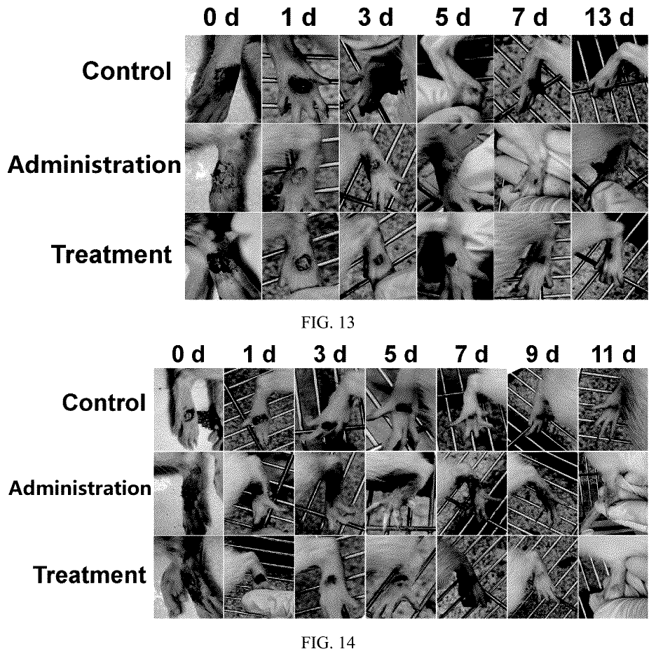

Therapeutic drug of epalrestat (M2 group) is applied daily on the foot gangrene disposed on the left hindfoot of the skin scratch model rat combined with glacial acetic acid, and then the wound (i.e., foot gangrene) of the model rat is observed. The wound of the model rat is shown in FIG. 13. There is no significant difference in healing speed between the foot treated with the epalrestat and the control group, and no acceleration in healing speed is observed.

Therapeutic drug of mecobalamin (H2 group) is applied daily on the foot gangrene disposed on the left hindfoot of the skin scratch model rat combined with glacial acetic acid.

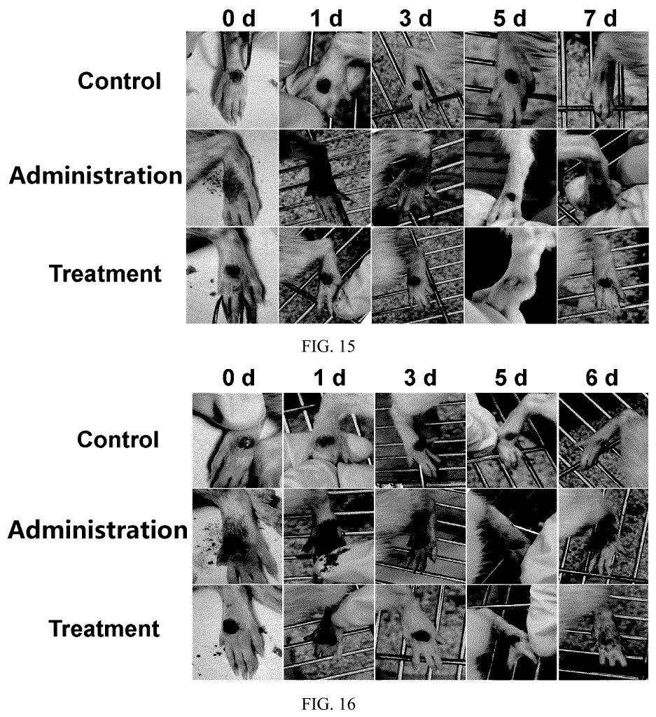

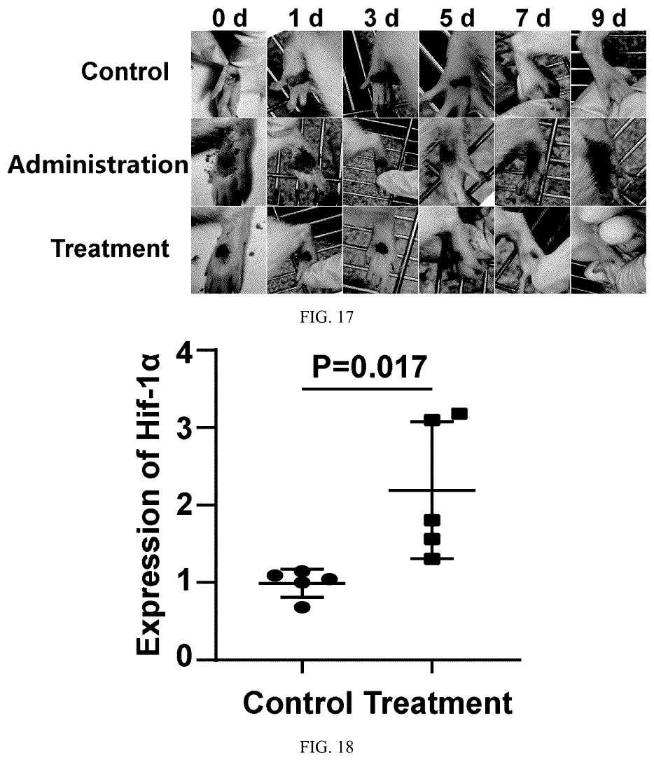

The hydrogels (T5 group, T6 group, T7 group, T8 group) are applied daily on the foot gangrenes disposed on the left hindfoots of the skin scratch model rats combined with glacial acetic acid and then the wounds (i.e., foot gangrenes) of the model rats are observed. The wound of the T5 group is shown in FIG. 14, the wound of the T6 group is shown in FIG. 15, the wound of the T7 group is shown in FIG. 16, and the wound of the T8 group is shown in FIG. 17. The above results show that the healing speed of the wounds treated with the hydrogel is similar to those of the control group, and the healing speed of the wound area is faster than that of the control group.

### 4. Effects of the Hydrogel on Cytokine Secretion

(1) Hif-1α Detection in Skin Tissues of the Rat Through Real-Time Quantitative Polymerase Chain Reaction (qPCR)

The following method is used to detect the content of Hif-1α in the skin tissues of the rat by using qPCR.

### 1) Primer Design

Primers used in the Hif-1α detection through qPCR are obtained from National Center for Biotechnology Information (NCBI) reference sequence: NM_022528.3 (i.e., Rattus norvegicus hypoxia inducible factor 1 subunit alpha (Hif1α), mRNA). The primers are designed by Primer-BLAST as a tool for finding specific primers. And the primers are synthesized by Shanghai Generay Biotechnology Co., Ltd; and the primers are illustrated in the following Table 4.

TABLE 4
Designed primer pairs used in the detection
by using qPCR

| Gene | | |
|---|---|---|
| Name | | Sequence |
| Hif1α | F | 5′-CTGGTGGCTCAGCAGTCTAT-3′ SEQ ID NO: 1 |
| | R | 5′-GAAATGCTGGGGAGCTGTGA-3′ SEQ ID NO: 2 |
| β- | F | 5′-CACTCCAAGTATCCACGGCA-3′ SEQ ID NO: 3 |
| Actin | R | 5′-TCCTCCCCTTAGGAGTTGGG-3′ SEQ ID NO: 4 |

### 2) Reagent and Consumables

The main reagents and consumables are illustrated in the following Table 5.

TABLE 5
Sources of main reagents of qPCR test

| Reagent/Consumable | | |
|---|---|---|
| material | Company | Item No |
| Chloroform | Shanghai Sangon | 80041667- |
| | Biotechnology Co., Ltd | 0.6*10 |
| Isopropyl alcohol | Shanghai Sangon | CAS.67-63-0 |
| | Biotechnology Co., Ltd | |
| Ethyl alcohol | Shanghai Sangon | CAS.64-17-5 |
| | Biotechnology Co., Ltd | |
| Trizol | Suzhoujunxin | M00501 |
| | Biotechnology Co., Ltd | |
| Diethylpyrocarbonate- | Shanghai Sangon | B501005-0100 |
| treated water (DEPC H₂O) | Biotechnology Co., Ltd | |
| Reverse transcription kit | Kangwei Century | Cat.CW2582M |
| | Biotechnology Co., Ltd | |
| 2x SYBR Green qPCR | Shanghai Donghuan | M00601 |
| Mastei Mix | Biotechnology Co., Ltd | |

### 3) Real-Time Fluorescence Quantitative PCR Detection of the Rat Tissues

The fluorescence quantitative PCR detection is performed according to the following steps, and a treatment group and a blank control group are set up.

RNA extraction: the Trizol illustrated in Table 5 is used to extract RNA from the skin tissues of the model rat, especially including the following steps (with reference to instructions of the corresponding reagent). The skin tissues are ground in to a fine powder by using liquid nitrogen, and then 1 mL of the Trizol reagent is added into the fine powder combined with blowing and splitting to obtain lysed cell fluid, and the lysed cell fluid is inhaled into an eppendorf (EP) tube with a volume of 1.5 mL. Thereafter, 200 μL of chloroform is added to the lysed cell fluid to obtain mixed solution, and then the mixed solution is shaken well and let stand until stratification appears; and then the stood mixed solution is centrifuged at 4° C. for 15 minutes with a speed of 12,000 revolutions per minute (rpm), thereafter obtaining supernatant. Furthermore, the isopropyl alcohol in ⅓ volume of the supernatant is added into the supernatant and the supernatant added with the isopropyl alcohol are mixed well, and then are placed in the −20° C. refrigerator and let stand for 20 minutes to obtain standing solution. Moreover, the standing solution is centrifuged at 4° C. for 10 minutes with a speed of 12,000 rpm, and then obtained supernatant is discarded to obtain lysate. 1 mL of 75% ethyl alcohol is added into the lysate to perform centrifugation at 4° C. for 10 minutes with a speed of 12,000 rpm, and obtained supernatant is discarded to obtain a crude product; and then 1 mL of anhydrous ethanol is added into the crude product to perform centrifugation at 4° C. for 10 minutes with a speed of 12,000 rpm (aiming at more conducive to subsequent drying) (noting that the RNA may float); and obtained supernatant is discarded again and the extracted RNA is dried; finally 30 μL DEPC H₂O (if a concentration of the extracted RNA is high, it can be diluted again) (same as RNase-free water), and the extracted RNA added with the DEPC H₂O is shaken slightly, and then is stored in the −80° C. refrigerator.

Reverse transcription: the reverse transcription kit illustrated in Table 5 is used to perform the reverse transcription on the extracted RNA (with reference to instructions of the kit for details). Specially, the reverse transcription is performed according to a system with a volume 20 μL; and then the system is diluted into three times of the volume by using the DEPC H₂O after the reverse transcription and is stored at −20° C.

qPCR detection: the 2× SYBR Green qPCR Mastei Mix illustrated in Table 5 is used for the qPCR detection (with reference to instructions of the reagent kit for details). Complementary DNA (cDNA) is taken with a volume of 2 μL; and the test is conducted according to the system with the volume of 20 μL.

The test results are illustrated in FIG. 18. The results show that the hydrogel for promoting growth of MSCs can promote the secretion of Hif1α.

### (2) Detection of Concentrations of VEGF and SDF-1α, and PDGF-β in Rat Serum by Using Enzyme Linked Immunosorbent Assay (ELISA).

Detection of concentrations of VEGF and SDF-1α, and PDGF-β in rat serum is performed by using ELISA, while setting up a treatment group and a blank control group. Main reagents used in the ELISA are illustrated in Table 6.

TABLE 6
Sources of the main reagents used in ELISA

| Reagent/Consumable | | |
|---|---|---|
| material | Company | Item No |
| Rat (VEGF) ELISA kit | Xiamen LunChangShuo | ED-30908 |
| | Biotechnology Co., Ltd | |
| Rat (SDF-1α) ELISA kit | Xiamen LunChangShuo | ED-30479 |
| | Biotechnology Co., Ltd | |
| Rat (PDGF-β) ELISA kit | Xiamen LunChangShuo | ED-34434 |
| | Biotechnology Co., Ltd | |

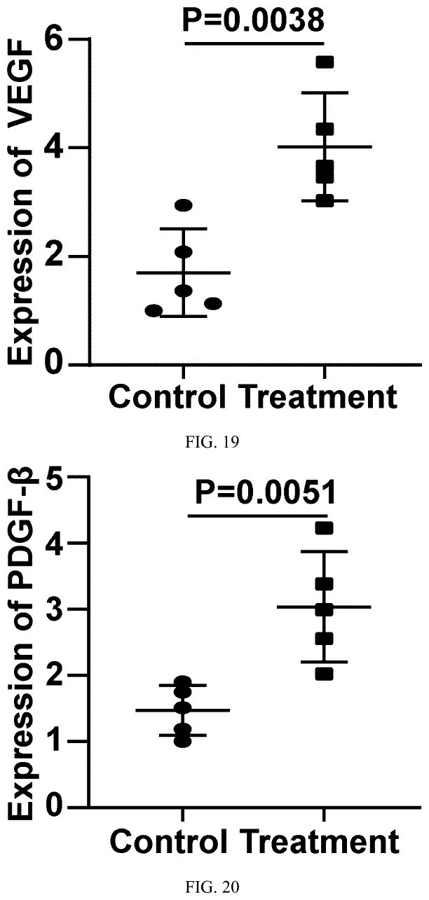

The rat (VEGF) ELISA kit illustrated in Table 6 is used to detect the concentration of VEGF in the rat serum (with reference to instructions of the kit for details). The test result is shown in FIG. 19. The result shows that the hydrogel for promoting growth of MSCs can promote the secretion of VEGF.

The rat (SDF-1a) ELISA kit illustrated in Table 6 is used to detect the concentration of SDF-1α (with reference to instructions of the kit for details). The test result is shown in FIG. 20. The result shows that the hydrogel for promoting growth of MSCs can promote the secretion of SDF-1α.

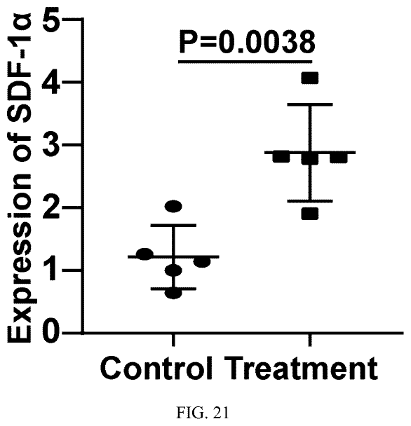

The rat (PDGF-β) ELISA kit illustrated in Table 6 is used to detect the concentration of PDGF-β (with reference to instructions of the kit for details). The test result is shown in FIG. 21. The result shows that the hydrogel for promoting growth of MSCs can promote the secretion of PDGF-β.

Finally, it should be noted that the above embodiments are only used to illustrate the technical solution of the disclosure and not to limit the disclosure. Although the disclosure has been described in detail with reference to the illustrated embodiments, those skilled in the related art should understand that the technical solution of the disclosure can be modified or equivalently replaced without departing from the purpose and scope of the technical solution of the disclosure. Moreover, the modifications and the equivalent replacements should be covered within the scope of the disclosure.

What is claimed is:

1. A preparation method of a hydrogel for promoting growth of mesenchymal stem cells (MSCs), comprising the following steps:
using natural raw materials to construct the hydrogel for promoting growth of MSCs, mixing the hydrogel with MSC-derived exosomes to obtain a sustained-release preparation, and administrating the sustained-release preparation on a wound affected with diabetic foot ulcer (DFU).

2. A hydrogel for promoting growth of MSCs, wherein the hydrogel for promoting growth of MSCs is obtained from the preparation method as claimed in claim 1.

3. An application method of the hydrogel for promoting growth of MSCs as claimed in claim 2, comprising: preparing a drug for healing skin affected with diabetic foot ulcer (DFU).

4. The application method as claimed in claim 3, further comprising:
up-regulating an expression of hypoxia inducible factor 1 subunit alpha (Hif-1α) in the DFU by using the drug prepared by the hydrogel for promoting growth of MSCs.

5. The application method as claimed in claim 4, further comprising:
accelerating secretions of vascular endothelial growth factor (VEGF), stromal cell-derived factor-1 (SDF-1α), and platelet derived growth factor-beta (PDGF-β) by using the drug prepared by the hydrogel for promoting growth of MSCs.


SEQUENCE LISTING (4 sequences)

SEQ ID NO: 1 - DNA, 20 bp, synthetic construct
  source 1..20: mol_type = other DNA
ctggtggctcagcagtctat

SEQ ID NO: 2 - DNA, 20 bp, synthetic construct
  source 1..20: mol_type = other DNA
gaaatgctggggagctgtga

SEQ ID NO: 3 - DNA, 20 bp, synthetic construct
  source 1..20: mol_type = other DNA
cactccaagtatccacggca

SEQ ID NO: 4 - DNA, 20 bp, synthetic construct
  source 1..20: mol_type = other DNA
tcctccccttaggagttggg